(19) United States
(12) Patent Application Publication
(10) Pub. No.: US 2009/0215670 A1
(43) Pub. Date: Aug. 27, 2009

(54) Site specific pegylated hemoglobin, method of preparing same, and uses thereof

(76) Inventors: Seetharama A. Acharya, Cresskill, NJ (US); Belur N. Manjula, Cresskill, NJ (US); Vivek N. Acharya, Cresskill, NJ (US)

Correspondence Address: AMSTER, ROTHSTEIN & EBENSTEIN LLP, 90 PARK AVENUE, NEW YORK, NY, 10016, US

(21) Appl. No.: 11/919,788

(22) PCT Filed: May 10, 2006
(86) PCT No.: PCT/US06/18037

§ 371 Date: Dec. 30, 2008

Related U.S. Application Data
(60) Provisional application No. 60/679,938, filed on May 11, 2005.

(51) Int. Cl. A61K 38/42; C07K 14/805

(52) U.S. Cl. 514/6; 530/385

(57) ABSTRACT

The present invention provides pegylated hemoglobins comprising a maleimide polyethylene glycol (PEG) conjugated to a thiol moiety of a cysteine residue of hemoglobin, methods of preparing the pegylated hemoglobins, compositions and blood substitutes comprising the pegylated hemoglobins, and methods of treating a subject which comprise administering to the subject blood substitutes comprising vasoinactive pegylated hemoglobins.

### CROSS-REFERENCE TO RELATED APPLICATION

The application claims the benefit of U.S. Provisional Patent Application No. 60/679,938, filed May 11, 2005, the content of which is hereby incorporated by reference.

### STATEMENT OF GOVERNMENT SUPPORT

This invention was made with United States government support under National Institutes of Health (NIH) grant numbers HL58247 and HL71064, and U.S. Army grant PR023085. Accordingly, the United States government has certain rights in this invention.

### BACKGROUND OF THE INVENTION

Throughout this application various publications are referred to in parenthesis. Full citations for these references may be found at the end of the specification immediately preceding the claims. The disclosures of these publications are hereby incorporated by reference in their entireties into the subject application to more fully describe the art to which the subject application pertains.

The development of blood substitutes as in vivo oxygen-carriers has been one of the major aspects of modern transfusion medicine (Winslow, 1999). Vasoactivity of acellular hemoglobin (Hb) has been the primary toxicity that needs to be modulated to realize the therapeutic benefits of Hb as an acellular oxygen carrier. The scavenging of the vasodilator nitric oxide (NO) by acellular Hb has been advanced as a molecular explanation for the vasoactivity of Hb (Motterlini et al., 1996). Accordingly, Hbs modified by site directed mutagenesis with reduced affinity towards nitric oxide and design of Hb derivatives with enhanced molecular size, such as the polymerized Hb, have been advanced as strategies for the development of non-hypertensive Hb based oxygen carriers. Both mutant Hbs with substitutions in the heme pocket and polymerized Hb appear to have reduced vasoactivity as compared to acellular Hb. Some of these modified Hbs are in clinical trials.

Polyethylene glycol (PEG) chains have been used to modify Hb. The observation by Rohlfs et al. (1998) that Enzon PEGylated bovine Hb is non-hypertensive, even though its NO binding activity is comparable to that of non-PEGylated modified forms of acellular Hbs that exhibit varying degrees of vasoactivity, suggested that PEGylation may be an approach to neutralize the vasoconstrictive activity of acellular Hb. Solutions of Enzon PEGylated bovine Hb are also distinct from solutions of other modified forms of Hbs that are vasoactive in that Enzon PEGylated bovine Hb solutions exhibit higher viscosity and higher colloidal osmotic pressure. Therefore, it has been suggested that PEGylation induced unusual solution properties of Enzon PEGylated Hb that may be responsible for the neutralization of the vasoactivity of acellular Hb (Vandegriff et al., 1997). Interestingly, these solution properties are also the properties of colloidal plasma volume expanders. Engineering the solution properties of colloidal plasma expanders, namely higher viscosity and colloidal osmotic pressure, to Hb by PEGylation could be a design strategy to develop Hb based oxygen carriers (Tsai and Intaglietta, 2001).

In an attempt to establish that PEGylation of human Hb can indeed facilitate the neutralization of its vasoactivity, a simplified procedure for PEGylation of proteins, thiolation mediated maleimide chemistry based PEGylation, has recently been developed (Acharya et al., 1996). Using this new PEGylation protocol, a hexaPEGylated Hb [(SP-PEG-5K)₆-Hb] has been generated, which was vasoinactive when transfused into hamsters and was nonhypertensive when infused into rats (Acharya et al., 2003). The non-hypertensive Enzon PEGylated Hb has been reported to carry ten copies of PEG-5K chains per tetramer (Vandegriffet al., 1997). Since thiolation mediated maleimide chemistry based PEGylation neutralizes the hypertensive activity of acellular Hb with six copies of PEG-5K chains, the higher number of PEG-5K chains present in the non-hypertensive Enzon PEG-Hb may reflect the role of the chemistry of PEGylation in neutralizing the vasoactivity of Hb. The conjugation chemistry used to generate the Enzon PEGylated bovine Hb links the PEG-chains directly to the ε-amino groups of surface lysine (Lys) residues through an urethane linkage and lacks the extension arm (δ-mercapto butyrimidyl chain) of the non-hypertensive hexaPEGylated human Hb of Acharya et al. (2003). The term “extension arm” refers to a carbon chain-thiol group that is attached to an amino group of hemoglobin. The extension arm places the thiol group away from the surface of hemoglobin, thereby enhancing the accessibility of the thiol group to bulky PEG reagents. The Enzon PEGylated bovine Hb results in neutralization of the positive charge of the ε-amino group of the surface Lys residue PEGylated and is thus a non-conservatively PEGylated molecule. In contrast, the HexaPEGylated-human Hb developed by Acharya et al. (2003) is a conservatively PEGylated Hb. Therefore, it follows that conservative PEGylation of Hb by the thiolation mediated maleimide chemistry based PEGylation neutralizes the vasoactivity of Hb with a lower number of PEG-5K chains than that present in the Enzon PEGylated bovine Hb.

The non-hypertensive hexaPEGylated Hb exhibits a high degree of molecular size homogeneity; however, it lacks chemical homogeneity. With as many as seven amino groups/αβ dimer being the reactive sites for thiolation mediated PEGylation, the reactivity of the various maleimide PEG reactive sites ranges from 12 to 100% . Consequently, one would anticipate the PEGylated Hb to be a mixture of species of PEGylated Hbs with the number of PEG-5K chains ranging from fourteen to two copies per tetramer. The molecular size homogeneity of the PEGylated Hb suggests that the product is probably a mixture of species with six and eight copies of PEG-chains per tetramer. The structural/conformational aspects of the hexa- and/or octaPEGylated Hb restricts the further PEGylation of these species. This suggests that more than one pattern of PEGylation of Hb to the stage of HexaPEGylation with maleimide PEG-5K can result in the neutralization of Hb.

### SUMMARY OF THE INVENTION

The present invention provides a PEGylated hemoglobin comprising a maleimide polyethylene glycol (PEG) conjugated to a thiol moiety of a cysteine residue of hemoglobin.

The invention also provides a method of preparing a PEGylated hemoglobin which comprises conjugating a maleimide polyethylene glycol (PEG) to a thiol moiety of a cysteine residue of hemoglobin.

The invention further provides compositions and blood substitutes comprising vasoinactive PEGylated hemoglobins and methods of treating a subject which comprise administering to the subject any of the vasoinactive PEGylated hemoglobins or blood substitutes disclosed herein or any vasoinactive PEGylated hemoglobin or blood substitute prepared by any of the methods disclosed herein.

Additional objects of the invention will be apparent from the description which follows.

### BRIEF DESCRIPTION OF THE FIGURES

FIG. 1. Size Exclusion Chromatography (SEC) analysis of canine Hb PEGylated with maleimide PEG-5K. The PEGylation of Hb was monitored as reflected by the enhancement in the molecular volume (hydrodynamic volume) of Hb by size exclusion chromatography of the reaction mixture. The canine Hb after incubation with maleidophenyl PEG-5000 for different periods of time has been analyzed.

FIG. 2A-2E. Ion exchange chromatographic purification of tetraPEGylated canine Hb on Q-Sepharose. FIG. 2A depicts the chromatographic pattern from Q-Sepharose column. FIG. 2B shows the SEC analysis of the major component eluting from the Q-Sepharose column and compares it with that of HbA, Di and hexaPEGylated Hb using maleimide PEG-5K, and DiPEGylated Hb using maleiemide PEG-10K. FIG. 2C depicts the RP-HPLC pattern of canine Hb (a) and PEGylated canine Hb (b) isolated by Q-Sepharose chromatography. FIG. 2D shows the SDS gel pattern of tetraPEGylated Hb and compares it with that of (SP-PEG5K)₂-Hb and (SP-PEG5K)₆-Hb. The tetraPEGylated canine Hb carries one major band with an apparent molecular weight of 27,000 and this mobility corresponds to that of human β-globin PEGylated with PEG 5K at its Cys-93. FIG. 2E depicts isoelectrofocusing pattern of PEGylated canine Hb. The isoelectric focusing pattern of a vasoinactive Hb generated using the thiolation mediated maleimide chemistry based PEGylation using maleimide phenyl PEG 5K is also shown for comparison.

FIG. 3A-3B. Viscosity and colloidal osmotic pressure of tetraPEGylated canine Hb. The studies are presented as a function of protein concentration and compared with that of (SP-PEG5K)₂-Hb and (SP-PEG5K)₆-Hb. A: Viscosity data and B: Colloidal osmotic pressure. Filled squares—canine Hb. Filled circles—(SP-PEG5K)₆-HbA. Filled triangles—TetraPEGylated canine Hb. (SP-PEG5K)₂-Hb.

FIG. 4. Reactivity of the animal Hbs with additional surface resides besides Cys-93(β) of HbA. The reactivity of feline, chicken, canine and human Hb have been compared.

FIG. 5A-5C. Molecular models of Hb A PEGylated at Cys-93(β) with maleimide phenyl PEG-5K (FIG. 5A); with maleimide PEG-10K (FIG. 5B), and a mutant of HbA where the residue 111 of the α-chain has been mutated to Cys residues and both Cys 111(α) and Cys-93(β) have been PEGylated using maleimide phenyl PEG-5K (FIG. 5C).

FIG. 6. Comparison of the physiological prorties of tetraPEGylated canine hB with that of hexaPEGylated Hb generated using thiolation mediated PEGylation protocol.

FIG. 7. Colloidal Osmotic Pressures (COP) of PEGylated DogHb (Δ) with 4 copies MalPhePEG3K compared with Dog Hb (□) and HbA () controls as a function of concentration. COP measured by Wescor 4420 Colloidal Osmometer in PBS (pH 7.4) at room temperature.

FIG. 8. Viscosity of PEGylated DogHb (Δ) with 4 copies MalPhePEG3K compared with Dog Hb (□) and HbA () controls as a function of concentration. Viscosity measurements were operated using the cone spindle (CPE-40, Brookfield) at a shear rate of 1125 per second in PBS (pH 7.4) at 37° C.

### DETAILED DESCRIPTION OF THE INVENTION

The present invention is directed to PEGylated hemoglobins, methods of preparing PEGylated hemoglobins, and uses of PEGylated hemoglobins, where the PEGylated hemoglobins are vasoinactive. As used herein, “PEGylation” means linking to polyethylene glycol (PEG), and a “PEGylated” hemoglobin is a hemoglobin that has PEG conjugated to it.

The invention provides a PEGylated hemoglobin comprising a maleimide polyethylene glycol (PEG) conjugated to a thiol moiety of a cysteine residue of hemoglobin. The maleimide polyethylene glycol (PEG) can be attached to a cysteine (Cys) residue, for example, at one or more of Cys-93(β), Cys-111(α), Cys-13(α), Cys-13(β), or Cys-130(α). Preferably, the PEGylated hemoglobin has two to eight (e.g., 2, 4, 6, or 8) polyethylene glycol (PEG) groups attached to the hemoglobin. More preferably, four polyethylene glycol (PEG) groups are attached to the hemoglobin. Preferably, each globin chain of the hemoglobin is PEGylated.

The maleimide PEG can be, for example, a maleimide PEG comprising an alkyl linker or, preferably, a maleimide phenyl PEG.

The PEGylated hemoglobin can comprise a PEG with a molecular weight of 200-40,000 daltons. Preferably, the polyethylene glycol (PEG) has a molecular weight of 3,000-5,000 daltons. Monomethyl PEGs of various molecular weights for use in PEGylating hemoglobin can be obtained commercially, for example from Nektar Therapeutics, CA.

Preferably, PEGylation of the hemoglobin does not alter the charge at the site of attachment to hemoglobin of the maleimide polyethylene glycol (PEG).

The PEGylated hemoglobins of the present invention do not require the use of an ‘extension arm’ to PEGylate the hemoglobin. As used herein, an “extension arm” refers to a carbon chain—thiol group that is attached to an amino group of the hemoglobin during a thiolation process, using for example 2-iminothiolane. An extension arm can, for example, comprise a δ-mercapto butyrimidyl chain or a γ-mercapto propylamide chain. The PEGylated hemoglobins of the present invention also do not require that a polyethylene glycol (PEG) chain be conjugated to an ε-amino group of a lysine residue of hemoglobin.

Preferably, PEGylated hemoglobins of the present invention are homogeneous both in terms of molecular size and sites of PEGylation.

Preferably, the PEGylated hemoglobins disclosed herein have a radius of 5-6 nm.

The PEGylated hemoglobins have a viscosity that is greater than non-PEGylated hemoglobin. Preferably, the PEGylated hemoglobins have a viscosity of at least 3 cp, i.e. a viscosity that is at least 3 times greater than the viscosity of non-PEGylated hemoglobin.

The PEGylated hemoglobins have a colloidal osmotic pressure that is greater than the colloidal osmotic pressure of non-PEGylated hemoglobin. Preferably, the colloidal osmotic pressure of the PEGylated hemoglobins is at least 4 (e.g., 4, 6, 8, 10 or 12) times greater than non-PEGylated hemoglobin.

The colloidal osmotic pressure of tetraPEGylated Hb is closer to that of (SP-PEG5K)₆-Hb previously produced using extension arms (Acharya et al., 2003). Accordingly, for the PEGylated hemoglobins of the present invention, a given mass of PEG conjugated to hemoglobin in the absence of extension arms can induce a larger increase in colloidal osmotic pressure than the same PEG mass in PEGylated hemoglobins that incorporate extension arms.

TetraPEGylated canine Hb maintains a good functional capillary density (70 to 80%) in extreme exchange transfusion (in the hamster model) and in this respect, it is comparable to previously prepared non-hypertensive hexaPEGylated Hb (Acharya et al., 2003). But, in the present invention, the same objective with respect to functional capillary density is achieved using nearly 35% less PEG conjugated onto Hb.

The PEGylated hemoglobin described herein can be prepared using hemoglobin of human, canine, chicken, sheep, murine, or feline origin.

As an example, in one preferred embodiment, the PEGylated hemoglobin is of canine origin and has a maleimide polyethylene glycol (PEG) conjugated to two Cys-93(β) residues and to two Cys-111(α) residues.

The invention provides methods of pegylating hemoglobin which comprise conjugating a maleimide polyethylene glycol (PEG) to a thiol moiety of a cysteine residue of hemoglobin. The method can use the various materials and parameters described herein

The invention further provides a PEGylated hemoglobin prepared by any of the methods disclosed herein.

The invention also provides a composition comprising any of the PEGylated hemoglobins disclosed herein or prepared by any of the methods disclosed herein, and a pharmaceutically acceptable carrier. The invention further provides a blood substitute comprising any of the PEGylated hemoglobins disclosed herein or prepared by any of the methods disclosed herein. Pharmaceutically acceptable carriers include, but are not limited to, saline, phosphate buffered saline, Ringer's solution, lactated Ringer's solution, Locke-Ringer's solution, Kreb's Ringer's solution, Hartmann's balanced saline solution, and/or heparinized sodium citrate acid dextrose solution. The pharmaceutical compositions also may comprise known plasma substitutes and plasma expanders. The pharmaceutical compositions of the present invention may be used as blood substitutes, and the like, and may be administered by conventional means including but not limited to transfusion and injection.

The invention provides a method of treating a subject which comprises administering to the subject any of the PEGylated hemoglobins or blood substitutes disclosed herein or any PEGylated hemoglobin or blood substitute prepared by any of the methods disclosed herein. Prior to treatment, the subject may have a reduced red blood cell count or a reduced blood volume. The reduced blood volume may be due to a wound or to surgery. The subject may have a disease characterized by vaso-occlusion or impaired blood flow. Such diseases include, but are not limited to, sickle cell disease, myocardial infarction and/or shock. Preferably, the treatment does not produce hypertension in the subject. Preferably, the treatment does not affect arteriolar diameter or venular diameter in the subject. For example, preferably the PEGylated hemoglobin does not cause constriction of arterioles. Preferably, the treatment does not increase vascular resistance in the subject. Preferably, the treatment does not affect the subject's heart rate.

The invention may be used in the treatment of human subjects or in veterinary medicine.

The present invention is illustrated in the following Experimental Details section, which is set forth to aid in the understanding of the invention, and should not be construed to limit in any way the scope of the invention as defined in the claims that follow thereafter.

### EXPERIMENTAL DETAILS

### I. Introduction

Homogeneity of a therapeutic product such as a blood substitute is a highly desirable quality, as the availability of homogeneous samples simplifies the task of correlating the structure of the therapeutic product with any toxicity. Engineering of a desired number of cysteine (Cys) residues on the surface of Hb (without perturbing its structure/conformation) by site directed mutagenesis as target sites for conjugating Maleimide PEG-chains is a simple strategy to generate homogeneous PEGylated Hb. This requires the identification of the potential sites in HbA to engineer the Cys residues.

Alternately, animal Hbs that carry additional reactive surface Cys residues as compared to human Hb can be used as substrates for generating homogeneous PEGylated Hbs that carry defined number of PEG-chains using maleimide chemistry without the use of iminothiolane for introduction of new thiol groups on the ε-amino group of surface Lys residues. Such PEGylated Hbs could also provide an insight into the potential role of the ‘extension arms’ present in non-hypertensive hexaPEGylated human Hb (Acharya et al., 2003). The engineering of the ‘extension arm’ between the ε-amino group of Lys and the PEG-chain and/or the conservation of the positive charge at the site of PEGylation appears to have increased the efficiency of the PEG-chains to neutralize the vasoactivity of acellular Hb. As described herein, the reaction conditions have been optimized to generate a tetraPEGylated canine Hb, and the PEGylation of Cys-111(α) and Cys-93 (β) of canine Hb has been shown to neutralize its vasoactivity. Furthermore, studies with other animal Hbs (feline Hb, murine Hb, and chicken Hb) have identified the sites α₁₃, α₁₁₁, α₁₃₀ and β₁₃ as potential positions for engineering maleimide reactive Cys residues into Hb to generate homogeneous PEGylated Hbs with the desired number of PEG-chains.

### II. Materials and Methods

Preparation of Hb: Human, canine, feline, chicken, and minor component of murine Hbs were prepared from lysates of their respective red blood cells (RBCs) by DE cellulose chromatographic procedures.

PEGylation of Hb and analysis of the PEGylated samples: Hb samples (0.5 mM) in phosphate buffered saline (PBS), pH 7.4 at 4° C., were reacted overnight with a 20 fold molar excess of maleimide phenyl PEG-5K over the reactive sulfhydryl groups of Hb. The reaction mixture was dialyzed against PBS buffer (three changes). A dialysis tubing with a 12 to 14,000 molecular weight cut off was used for dialysis. The dialyzed samples were concentrated by ultra-filtration to 32 mg/ml (˜0.5 mM) and analyzed by analytical size exclusion chromatography.

The relative reactivity of the surface cysteine (Cys) residues of animal Hb was compared by reacting respective animals Hbs (0.5 mM) at pH 7.4 with 2.5 mM maleimide phenyl PEG-5K for four hours and then analyzing the material by size exclusion chromatography.

Purification of PEGylated Hb: Chromatographic purification of the PEGylated sample was carried out by ion exchange chromatography on a Q-Sepharose HP column using an AKTA Purifier 10 system. Canine Hb reacted with the maleimido phenyl PEG5K was first dialyzed extensively against PBS, pH 7.4, followed by dialysis against 50 mM Tris-Ac, pH 8.5, the starting buffer for the ion exchange chromatography. The protein was eluted from the column using a linear gradient generated by 50 mM tris-acetate buffer pH 8.5 and 50 mM tris-acetate buffer pH 7.0. The column was eluted at a flow rate of 1.5 ml/min. The elution of the protein was continuously monitored at 240, 540 and 600 m. The major peak was pooled and subjected to further structural and functional studies.

Viscosity measurements: The viscosity of the PEGylated Hbs was measured in a cone and plate Rheometer (Brookfield, Middleboro, Mass.), as a function of the concentration of PEGylated Hb, in PBS buffer, pH 7.4 and at 37° C. The instrument was calibrated with deionized water prior to measurements of the viscosity of the Hb samples.

Colloidal osmotic pressure measurements: The colloidal osmotic pressure (COP) of the PEGylated Hbs was determined using a Wescor 4420 Colloidal Osmometer. Measurements were done as a function of the PEGylated Hb concentration, in PBS, pH 7.4 at room temperature. A 30 kDa MW cut-off membrane was used. The instrument was tested with Osmocoll reference standards prior to measurements of the samples.

Molecular radius: The molecular radius of the PEGylated Hbs was determined by dynamic light scattering measurements using an instrument from Protein Solutions, Inc., Model Dynapro MS/X.

Molecular modeling of PEGylated Hbs: Models of PEGylated Hbs were generated as described previously (Manjula et al. 2003).

Vasoactivity of PEGylated Hbs: Analysis of the vasoactivity and microvascular hemodynamics of the PEGylated Hb were carried out in a hamster skin fold window microcirculation model, essentially according to procedures previously described (Kerger et al. 1996, Mirhashemi et al. 1988, Tsai et al. 1995, 1996, 1998).

Analysis of the globin Chains: The α- and β-globin chains of PEGylated Hb conjugate were analyzed by RPHPLC using a Vydac C4-column.

### III. Results

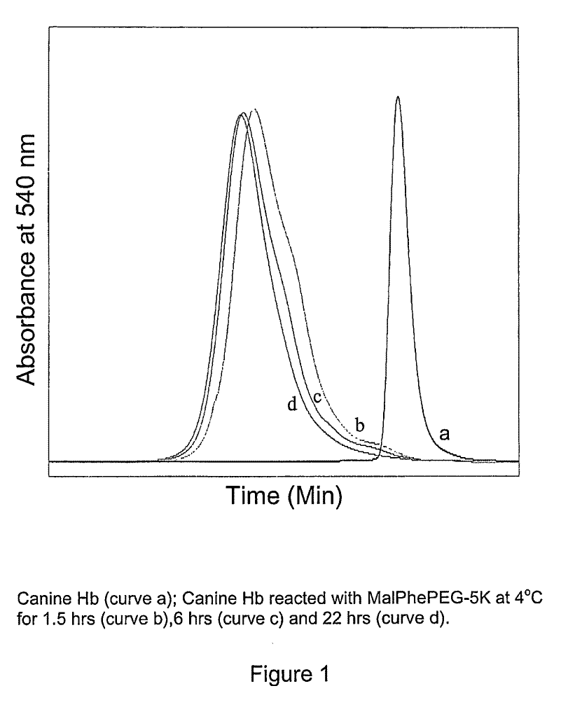

TetraPEGylation of canine Hb PEGylated using maleimide phenyl PEG-5000: The influence of incubation of canine Hb with a 20 fold molar excess of PEG-5K on the hydrodynamic volume of the protein is shown in FIG. 1. The canine Hb is readily PEGylated by maleimide PEG-5K. On incubation of canine Hb for 90 minutes, very little unmodified Hb remains in the reaction mixture. The protein elutes at a position corresponding to that of (SP-PEG10K)₂-Hb that carries a total PEG mass of 20,000 as two chains conjugated to Cys-93(β) of HbA. Given the similar hydrodynamic volume of the PEGylated canine Hb and DiPEGylated Hb with two copies of PEG-10 K conjugated, the PEGylated canine Hb appears to be tetraPEGylated Hb, with four copies of PEG-5K. The sample carries a shoulder which elutes at the position corresponding to the hydrodynamic volume of (SP-PEG5K)₂-Hb, suggesting the intermediate in the tetraPEGylation of canine Hb. As the time of incubation increases, the shoulder disappears with a slight shift in the peak position of the main peak. By six hours of reaction most of the shoulder disappears, but an overnight incubation is required to completely eliminate the shoulder. The generation of PEG-conjugated canine Hb on incubation of canine Hb (0.5 mM) in PBS with a 25 to 40 fold molar excess of maleimide phenyl PEG-5,000 has been chosen for the large scale preparation of tetraPEGylated Hb.

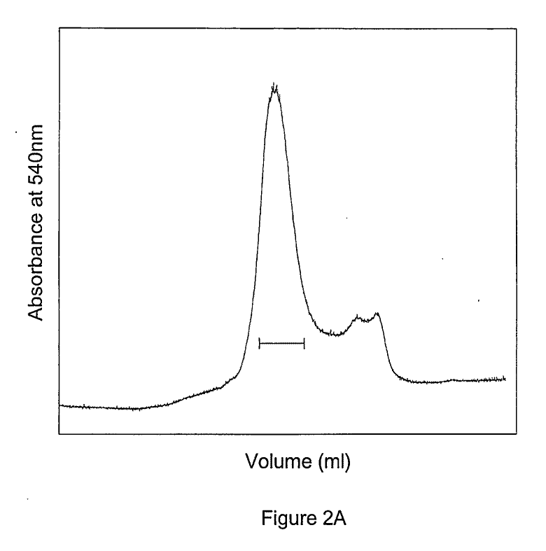

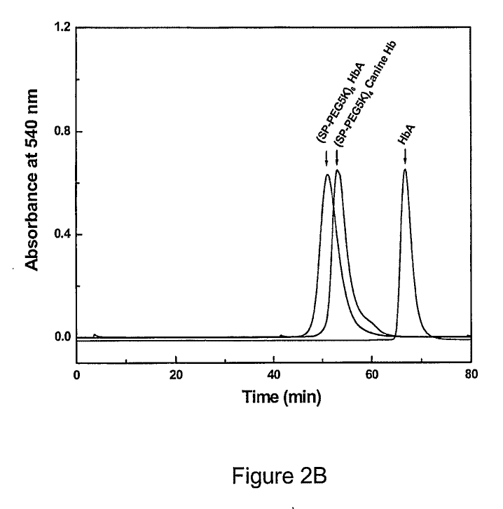

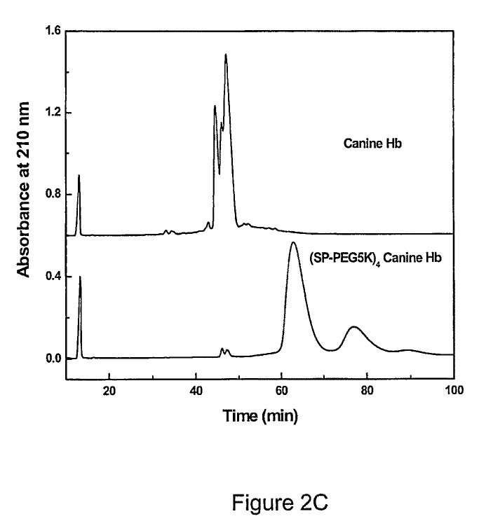

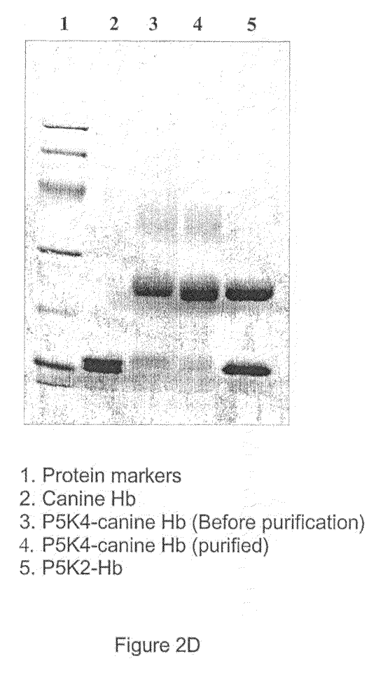

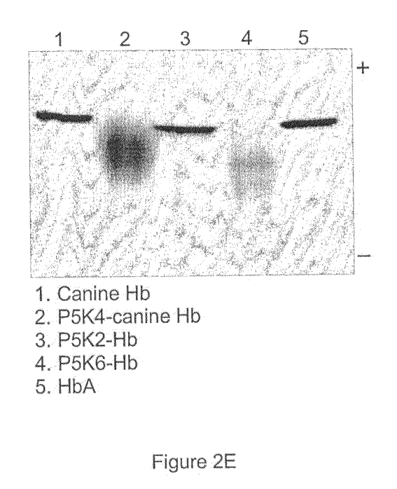

Chromatographic purification of the tetra PEGylated Hb: The ion exchange chromatographic pattern of tetra PEGylated canine Hb on a Q-Sepharose column is shown in FIG. 2A. The chromatographic profile of PEGylated canine Hb shows one major component and two minor components eluting slightly after the major component. The major component of the PEGylated canine Hb was pooled and concentrated to a 6 g % solution.

Characterization of the TetraPEGylated canine Hb: The hydrodynamic volume of the tetraPEGylated canine Hb has been compared with that of other PEGylated human Hbs, site specifically PEGylated at Cys-93(β) with PEG-5K, PEG-10K and PEG-20K, respectively, using size exclusion chromatography (FIG. 2B). The PEGylated canine Hb exhibits a hydrodynamic volume that is slightly lower, as compared to that of (SP-PEG5K)₆-Hb.

The HPLC pattern of the Q-Sepharose purified PEGylated canine Hb is shown in FIG. 2C. There is very little unmodified globin chains in the Q-Sepharose purified sample suggesting the near complete modification of the globin chains. The PEGylated Hb carried two components (and a trace amount of the third one), the major one accounting for more than 80% of the sample, and minor one that eluted later than the major fraction. Trace amounts of a third component eluted after the minor components. The mass spectral data suggested that the major component corresponds to globin chains with one copy of the PEG-5K chain conjugated to the globin chain, and the second minor component carries two PEG-5K chains per globin chain. The results suggest that the maleimide PEG-5K reacts at some sites in addition to the Cys-residues at α₁₁₁ and β93 of canine Hb, presumably at the α-amino groups of either of α-chain or of β-chain or of both chains.

SDS-PAGE analysis of the Q-Sepharose purified PEGylated canine Hb is shown (FIG. 2D). Almost all of the globin chains moved with an apparent molecular weight of 27,000 daltons. There is an absence of unmodified globin chains, and the presence of one major band with an apparent molecular mass of 27,000. This pattern has been compared with that of (SP-PEG-5K)₂-Hb that carries a PEG-5K chain conjugated to the β-chain at Cys-93(β). This comparison shows that the protein band with an apparent molecular size of 27,000 dalton present in PEGylated Hb corresponds to the monoPEGylated globin chain present in previously studied DiPEGylated Hb. The results therefore demonstrate that both the α- and the β-chains of canine Hb predominate as the monoPEGylated derivatives. This result identifies this PEGylated Hb as the tetraPEGylated canine Hb, each globin chain of the PEGylated protein carrying one PEG5K chain. There may be noted trace amounts of unmodified globin chains as well as trace amounts of modified globin chains with molecular weights higher than that of monoPEGylated globin chains. This protein band corresponds to the PEGylated band present in the sample (SP-PEG-10K)₂-Hb (data not shown) that has β-chain PEGylated with PEG-10K at its Cys-93(β). Accordingly, it is concluded that this peak carries globin chains conjugated with two copies of PEG-5K chains. This is consistent with the molecular size homogeneity of the sample, and reflects the reactivity of maliemide PEG-Hb at sites other than Cys-111(α) and Cys-93(β).

The isoelectric focusing gels (FIG. 2E) have established that the tetraPEGylated canine Hb is devoid of any unreacted Hb. However, the PEGylated sample electrofocussed as a diffused band, whereas the diPEGylated Hb [that has the PEGylation only on Cys-93(β)] electrofocussed as a sharp band. The hexaPEGylated Hb generated by the thiolation mediated maleimide chemistry based PEGylation also electrofocussed as a diffused band. Thus, the diffused isoelectric focusing pattern of the tetraPEGylated canine Hb appears to be a consequence of PEGylation of Hb with multiple copies of PEG-chains.

The molecular radius of canine Hb is 3.1 nm and is comparable to that HbA (3.12 nm). The tetra-PEGylated canine Hb exhibits a molecular radius of 5.32 nm (Table I). This value is very close to that of (SP-PEG10K)₂-Hb (5.54 nm), but noticeably smaller than that of the hexaPEGylated Hb [(SP-PEG5K)₆-Hb]. The molecular radius of the latter is around 6.5 nm. The molecular radius determined by light scattering very closely parallels the hydrodynamic volume as reflected by size exclusion chromatography. Thus size enhancement seen on tetraPEGylation of canine Hb is smaller than that seen on hexaPEGylation of human Hb with PEG-5K, and slightly smaller than that seen on diPEGylation of human Hb with PEG-10K.

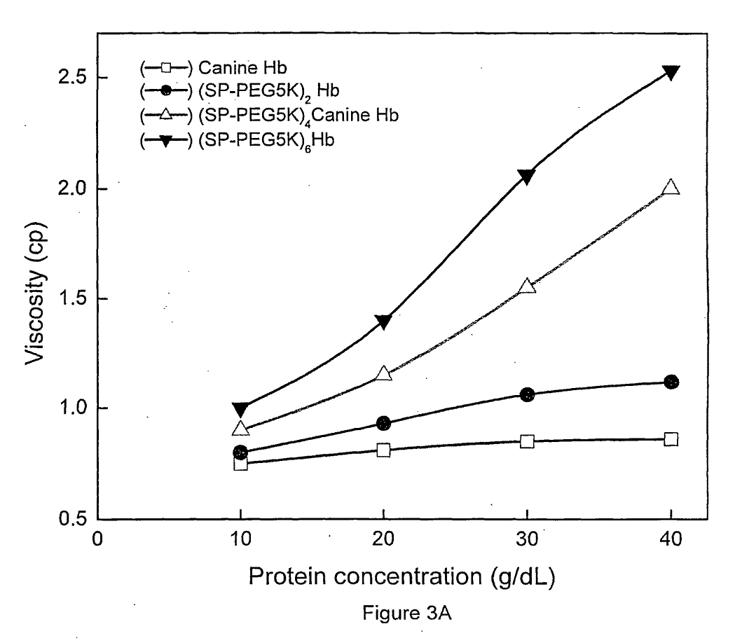

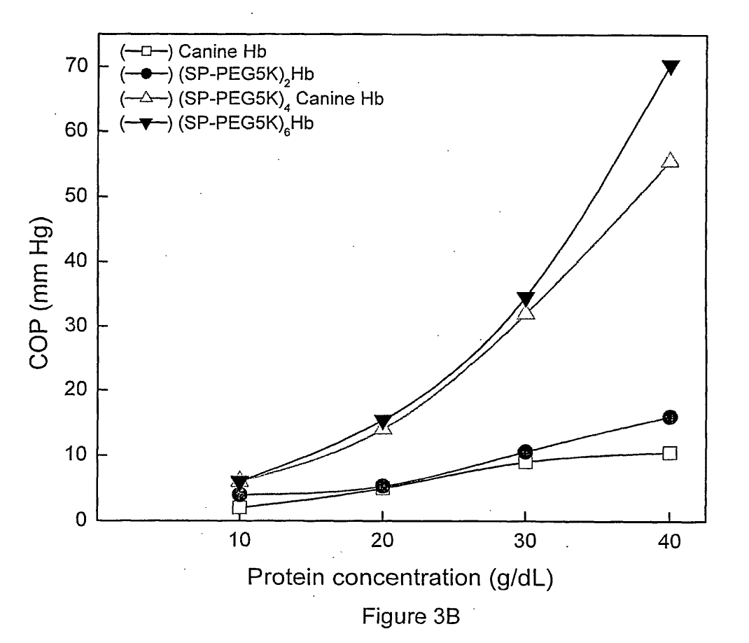

Viscosity and the colloidal osmotic pressure of tetra-PEGylated Hbs: The viscosity of the tetra-PEGylated canine Hb in PBS, pH 7.4 at 37° C. as a function of protein concentration is presented in FIG. 3A and compared with that of (SP-PEG5K)₆-Hb and control canine Hb. The viscosity of the PEGylated Hb increases in a slight exponential fashion when studied as a function of the protein concentration. Even though the tetraPEGylated canine Hb carries only four copies of PEG-5K chains per tetramer, its viscosity is only slightly lower than that of (SP-PEG5K)₆-Hb, which carries six copies of PEG-5K chains per tetramer at all the protein concentrations studied. However, the viscosity of tetraPEGylated canine Hb is significantly higher than that of (SP-PEG5K)₂-Hb suggesting that the increase in the viscosity of Hb on conjugation with PEG-5K is not a direct correlate of the number of PEG-5K chains per tetramer.

The colloidal osmotic pressure of tetra-PEGylated canine Hb is presented in FIG. 3B as a function of protein concentration and compared with that of hexaPEGylated and diPEGylated Hb. As the concentration of the tetraPEGylated canine Hb and hexaPEGylated Hb is increased, the colloidal osmotic pressure increases in an exponential fashion. On the other hand, the conjugation of two copies of PEG-5K to Hb has very little influence on the colloidal osmotic pressure of Hb when studied as a function of protein concentration. Interestingly, the colloidal osmotic pressure of tetraPEGylated Hb is comparable to that of (SP-PEG5K)₆-Hb when studied as a function of protein concentration. The conjugation of two copies of PEG-5K to Cys-93(β) and four copies of PEG-5K to the amino groups of human Hb using an ‘extension arm’ appears to have nearly the same influence as the conjugation of four copies of PEG-5K to the two copies of Cys-111(α) and Cys-93(β) of canine Hb without using ‘extension arms’. Conjugating the PEG-chains to the —SH groups of Cys residues appears to be a more efficient approach to endow colloidal osmotic pressure to Hb. Accordingly, the results suggest that the chemistry of conjugation influences the physical properties of the solutions of PEGylated Hb.

Comparison of the systemic and microvascular responses of TetraPEGylated canine Hb with canine Hb: The influence of conjugating four copies of PEG-5K onto canine Hb on the micro circulation has been evaluated in the hamster skin fold window model by comparing the acute systemic and micro vascular response to a 10% hypervolemic infusion of tetraPEGylated Hb with that of unPEGylated canine Hb. Although both Hb solutions increased the mean arterial pressure immediately on infusion as compared to the hexaPEGylated Hb, the mean arterial pressure when infused with tetraPEGylated Hb remained nearly constant, at around 110% of the baseline throughout the period of the experiment. The control animal infused with canine Hb, on the other hand showed a maximum increase by thirty minutes after infusion, and this returned to the base level by sixty minutes after the infusion. The mean arterial pressure of the animal infused with (SP-PEG5K)₆-Hb remained at ˜105% of the baseline all through the period of observation.

The arteriolar vessels remained significantly constricted throughout the period after infusion of unPEGylated canine Hb. On the other hand no constriction was observed in the animals infused with tetraPEGylated canine Hb. Interestingly, they remained slightly dilated compared to the control samples. In the animals that were infused with (SP-PEG5K)₆-Hb, the arteriolar diameter remained close to the baseline values.

The functional capillary density decreased significantly on infusion with the canine Hb. But, in animals infused with tetraPEGylated Hb, the functional capillary density remained close to the baseline except for a slight increase at 10 minutes. The above studies of the arteriolar diameter and functional capillary density, after infusion of tetraPEGylated Hb, indicate that a vasoinactive PEGylated Hb can be generated by decorating Hb with four copies of PEG-5K chains, one on each of the four chains of the tetramer.

Measurements of the mean arterial pressure, arteriolar diameter and functional capillary density on infusion with hexaPEGylated Hb revealed that the tetraPEGylation of canine Hb appears to function nearly as well as the hexaPEGylation of human Hb in neutralizing the vasoconstrictive activity of the acellular Hb.

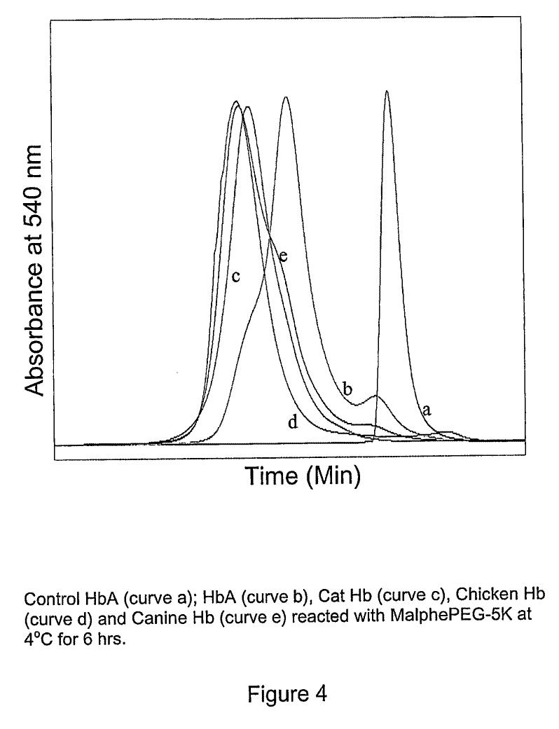

Comparison of the reactivity of the surface Cys residues of animal Hbs for PEGylation with maleimido phenyl PEG 5K: Many other animal Hbs that carry additional surface Cys residues could be used as candidates for generating PEGylated Hb with multiple copies of PEG-chains using maleimide phenyl PEG-5000; some of these are tabulated in Table II. The relative reactivity of the additional Cys residues of these animal Hbs for PEGylation with maleimide phenyl PEG-5K has been investigated in oxy conformation (FIG. 4). The PEGylation has been carried out using 12.5 mM PEG maleimide and a Hb concentration of 0.5 mM. With human Hb, this represents a 12.5 fold molar excess of PEG reagent over the reactive Cys residues. The presence of a new surface cys residue in the α-chains of canine and chicken Hb (as compared to the absence of these in human Hb) and in the β-chain of murine Hb makes the amount of PEG reagent used in these experiments 6.25 fold molar excess over the reactive Cys residues in these animal Hbs. In the case of feline Hb that carries two reactive Cys-residues in its α-chain, the amount of the PEG maleimide used represents a limiting concentration.

The presence of additional reactive Cys residues [besides Cys-93(β)] in Hbs of canine, chicken, and feline Hb is expected to facilitate the formation of tetraPEGylated Hb, if the reaction of the second reactive Cys in the αβ-dimer increases the propensity of these Hbs to generate tetraPEGylated Hb. Canine and feline Hbs were completely modified at their reactive Cys residues. However, chicken Hb, with its second reactive Cys at α₁₃₀ per, was not completely conjugated to generate tetraPEGylated Hb; some amount of diPEGylated Hb appears to be present in this sample. This suggests that the reactivity of Cys-130(α) is lower than that of Cys-111(α) of canine Hb. It may be noted that under these reaction conditions, even human Hb generated some amounts of tetraPEGylated Hb. This suggests that the specificity of the reaction of maleimide PEG to thiol groups is not absolute and that some reactions occur at other functional groups (either α or ε-amino groups of the protein are the potential sites). Such side reactions may be responsible for the larger hydrodynamic volume of the PEGylated Hbs generated by chicken Hb relative to the tetraPEGylated canine Hb.

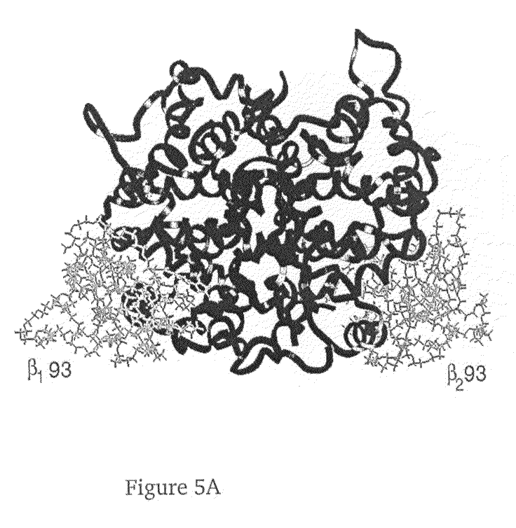

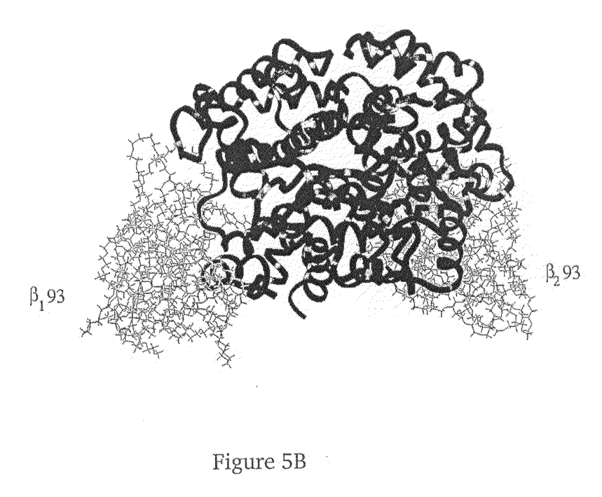

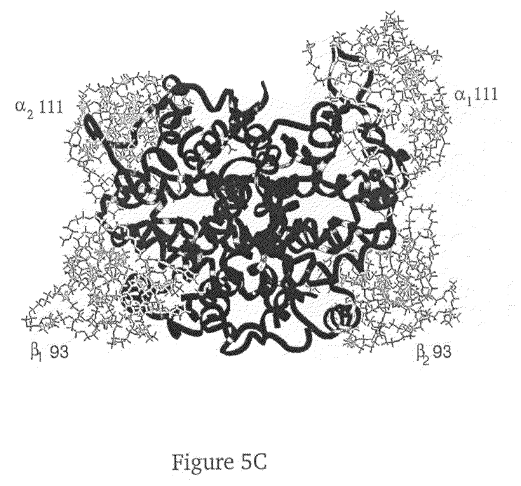

Molecular model of Tetra-PEGylated Hb: Molecular models of Hb site specifically PEGylated Hb at Cys-93(β) have previously been developed (Manjula et al., 2003). Ala-111(α) of human Hb has now been mutated through molecular modeling and PEGylated with maleimide PEG PEG-5K at Cys-93(β) and Cys-111(α). The generated molecular model is shown in FIG. 5 and compared with that of human Hb PEGylated at Cys-93(β) with maleimide PEG-SK and with PEG-10K. The tetraPEGylation of Hb with PEG5K increased the loss of the accessible molecular surface of Hb relative to diPEGylation of Hb with PEG5K or with PEG-1 OK (Table III). Thus, it is clear from the molecular modeling studies that surface decoration of Hb with four copies of PEG 5K conjugated at Cys-93(β) and Cys-111(α) of Hb gives a better surface coverage than conjugating two copies of PEG-10 K chains at Cys-93(β).

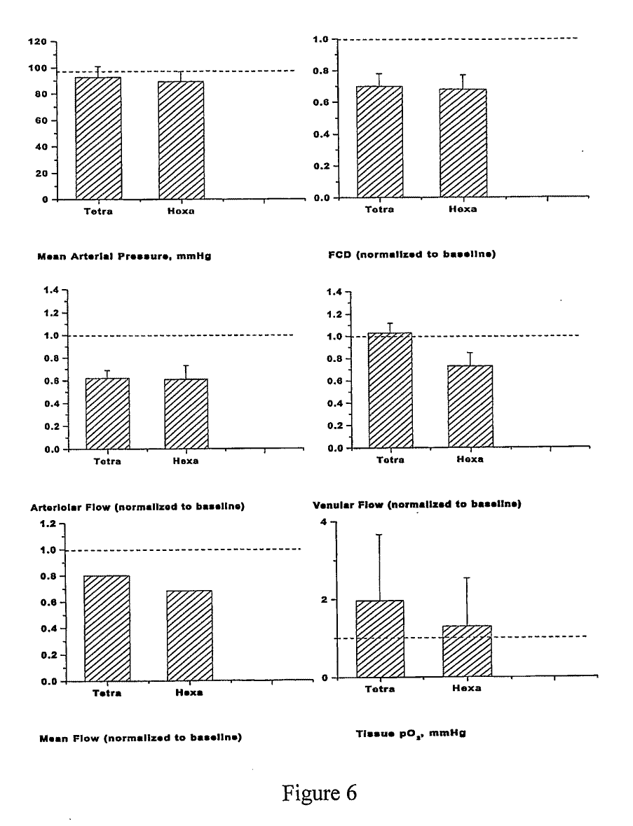

Tissue oxygenation by tetraPEGylated canine Hb: Given the fact that tetraPEGylated canine Hb is not vasoconstictive in top load studies, tissue oxygenation was also investigated using a 4 gm % solution of tetraPEGylated canine Hb in 50% exchange transfusion in hamster. The results are compared with that of hexaPEGylated Hb generated by thiolation mediated PEGylation protocol. As can be seen from FIG. 6, almost all physiological properties of tetraPEGylated Hb are comparable to that of hexaPEGylated Hb including tissue oxygenation. Accordingly, tetraPEGylation of Hb will be more than adequate for overcoming the vasoactivity of acellular Hb. This can reduce the cost of production of PEGylated Hb as the blood substitute.

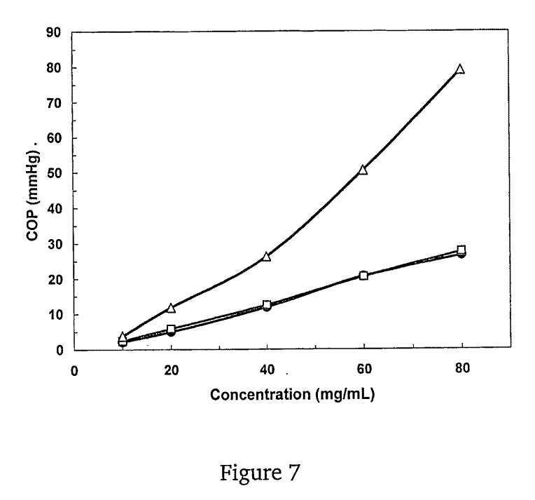

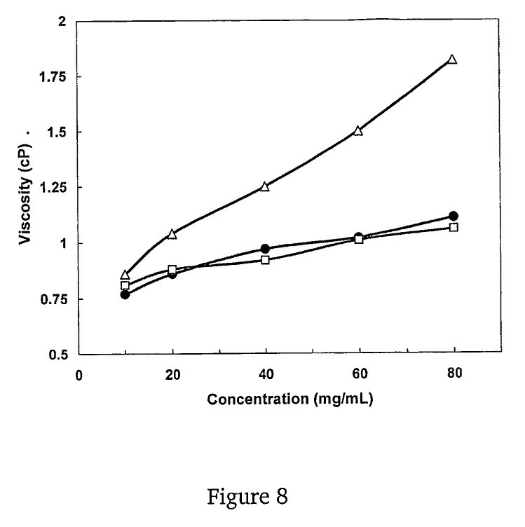

TetraPEGylation of canine Hb using maleimido phenyl PEG-3K: The hexaPEGylated Hb generated by thiolation mediated PEGylation as well as the tetraPEGylated canine Hb are high oxygen affinity species. The new paradigms for the design of Hb based oxygen carriers invoke the generation of high oxygen affinity products to minimize the release of oxygen from oxyPEGylated Hbs in the arterial side of the circulation so that auto-regulatory mechanisms of vaso-constriction are not triggered as a result of over supply of oxygen. However, it may be questioned whether such a product can deliver enough oxygen to the tissue. One way of supplying more oxygen to the tissues is to transfuse larger amounts of PEGylated Hbs, but the higher viscosity and COP of tetraPEGylated Hb generated using maleimido phenyl PEG-5K makes it difficult to infuse more concentrated tetraPEGylated canine Hb. To overcome this situation, tetraPEGylated canine Hb was prepared using maleimido phenyl PEG-3K. The viscosity and colloidal oncotic pressure of this material as a function of protein concentration is shown in FIGS. 7 and 8. This product can be used at a concentration of 8 gms per/dl, and in principle this could deliver twice the amount of oxygen to the tissues as compared to the solution of 4 gm % of tetraPEGYlated Hb prepared using maleimido phenyl PEG-5K.

TABLE I
Size Enhancement of Hb as a consequence of PEGylation.

| | Molecular Radius | Molecular volume |
|---|---|---|
| Sample | (nm) | (nm³) |
| HbA | 3.12 | 127 |
| Canine Hb | 3.10 | 124 |
| (SP-PEG5K)₂-Hb | 4.20 | 309 |
| (SP-PEG5K)₄-canine Hb | 5.34 | 636 |
| (SP-PEG10K)₂-Hb | 5.54 | 710 |
| (SP-PEG5K)₆-Hb^{a} | 6.51 | 1152 |

^{a}PEGylation carried out in the presence of 2-iminothiolane.

TABLE II
Reactive Cysteine residues of animal Hbs as potential
substrates for generation of homogeneous PEGylated
Hbs using maleimide phenyl PEG5K.

| | Number of Reactive | | |
|---|---|---|---|
| | Cysteines in oxy | | Expected PEGylated |
| Hemoglobin | conformation | Position | Hemoglobin |
| Human Hb | 2 | Cys-93(β) | (SP-PEG5K)₂-Hb |
| Canine Hb | 4 | Cys-111(α) | (SP-PEG5K)₄-Hb |
| | | Cys-93(β) | |
| Chicken Hb | 4 | Cys-130(α) | (SP-PEG5K)₄-Hb |
| | | Cys-93(β) | |
| Sheep Hb | 4 | Cys-111(α) | (SP-PEG5K)₄-Hb |
| | | Cys-93(β) | |
| Murine Hb | 4 | Cys-13(β) | (SP-PEG5K)₄-Hb |
| | | Cys-93(β) | |
| Feline Hb | 6 | Cys-13(α) | (SP-PEG5K)₆-Hb. |
| | | Cys-111(α) | |
| | | Cys-93(β) | |

TABLE III
Influence of PEGylation of Hb on Molecular Surface Accessibility

| | Loss of Accessible surface (A⁰)² |
|---|---|
| (SP-PEG5K)₂-Hb | 2388.0 |
| (SP-PEG5K)₄-canine Hb | 6312.3 |
| (SP-PEG10K)₂-Hb | 2600.0 |

### IV. Discussion

The results presented herein demonstrate that tetraPEGylation of canine Hb neutralizes its vasoconstrictive activity when assayed by the top load studies in the hamster window models. PEGylation of canine Hb using maleimide PEG-5K does not alter the charge at the site of the attachment. An earlier version of vasoinactive PEGylated Hb is the hexaPEGylated Hb, generated by thiolation mediated maleimide chemistry based PEGylation (Acharya et al., 2003), which also achieves PEGylation without altering the positive charge of the amino groups at the PEG-chain conjugating sites. The hexaPEGylated-Hb carries nearly 40% less PEG mass as compared to Enzon PEG-Hb, which carries ten copies of PEG-5K. A modified version of this hexaPEGylated Hb is MP4 (Vandegriff et al., 2003), the preparation of which also employed the thiolation mediated maleimide chemistry based PEGylation protocol developed by Acharya et al. (1996, 2003). Although the hexaPEGylated Hb exhibits molecular size homogeneity, it is not chemically homogeneous. The PEG-chains are distributed on multiple surface amino groups; the average number of PEG-chains distributed on the Hb tetramer turns out to be around six copies per chain. The present demonstration that the homogeneous tetraPEGylated canine Hb is vasoinactive, along with the observation that other animal Hbs could also be used to generate homogeneous tetraPEGylated Hb, opens the opportunity to design multiple nonhypertensive oxygen carrying plasma expanders that carry total PEG-mass lower than that in the hexaPEGylated Hb that is currently under clinical trial.

TetraPEGylated canine Hb is generated using maleimide chemistry, and does not involve the thiolation step since intrinsic thiol groups of Cys residues of Hb are PEGylated. Accordingly, in generating this tetraPEGylated canine Hb, as compared to the hexaPEGylated Hb generated by iminothiolane mediated maleimide chemistry based PEGylation, the ‘extension arm’ present in the non-hypertensive hexaPEGylated Hb between the side chain of Lys and the PEG-chain is absent in the tetraPEGylated Hb. Since the intrinsic Cys residues are the only sites of PEGylation, the PEGylation pattern in the tetraPEGylated canine Hb is very site specific and accordingly the pattern of PEGylation (surface decoration) in this vasoinactive PEG-Hb conjugate is very distinct as compared to that of hexaPEGylated Hb. Accordingly, it follows that four copies of PEG-5K chains conjugated to Hb through the thiols of Cys-111(α) and Cys-93(β) endow the canine Hb with solution properties sufficient to neutralize its vasoactivity and/or provides an adequate molecular shielding to make it vasoinactive.

An interesting aspect of this study is that although the viscosity of the tetraPEGylated canine Hb is lower than that of the hexaPEGylated Hb, the colloidal osmotic pressure of tetraPEGylated Hb is closer to that of the hexaPEGylated Hb. The four copies of PEG-5K chains of tetraPEGylated canine Hb are conjugated to the protein directly onto the thiol moieties of Cys-residues without the use of the ‘extension arms’ on the ε-amino groups that are present in the nonhypertensive hexaPEGylated Hb (Acharya et al., 2003). Since Cys-93(β) is completely PEGylated in both tetraPEGylated and hexaPEGylated Hb without the extension arm, it follows that four PEG-chains linked to the surface amino groups of Hb enhance the colloidal osmotic pressure of diPEGylated Hb nearly to the same extent as the two PEG-5K chains introduced onto the Cys 111 of canine Hb. The PEG-chains linked through the —SH groups of reactive Cys residues of canine Hb are expected to be closer to the molecular surface of Hb than those of the hexPEGylated Hb that are conjugated at the Lys residues. The PEG chains of the hexaPEGylated Hb generated by thiolation mediated PEGylation are expected to be placed away from the protein backbone through the longer side chains of Lys (compared to Cys residues) and the ‘extension arms.’ The colloidal osmotic pressure induced to Hb for a given mass of PEG conjugated to the Hb appears to be reduced by nearly 50% when the PEG chain is conjugated to the ε-amino groups of Hb through the ‘extension arm’ as compared to the PEG attached to the thiol group of a Cys residue. The larger influence of PEG chain conjugated to Cys is apparently a consequence of the closer packing of the PEG5K chain onto the molecular surface of Hb.

The tetraPEGylation of canine Hb has essentially neutralized its vaso-constrictive activity. A comparison of the molecular models of (SP-PEG5K)₂-Hb, and (SP-PEG5K)₄-Hb shows that tetraPEGylation does not provide the complete coverage of the molecular surface and calculation of surface area covered on diPEGylation and tetraPEGylation of Hb suggests that the addition of two more copies of PEG5K-chains on (SP-PEG5K)₂ nearly doubles the surface coverage of Hb. But the exponential dependence of the viscosity and colloidal osmotic pressure of tetraPEGylated canine Hb as the protein concentration increases suggests that inter molecular interactions of the tertaPEGylated canine Hb may be possible when the PEGylated Hb is placed in a crowded medium (as in the circulation).

Measurements of the functional capillary density of hamsters infused with PEGylated Hbs demonstrated that the three PEGylated Hbs, namely, tetraPEGylated canine Hb, hemoglobin diPEGylated with maleimide phenyl PEG-10K, and hexaPEGylated Hb, perform well in this assay. Accordingly these Hbs can be considered as potential oxygen carrying plasma expanders.

Given the propensity of the tetraPEGylated canine Hb to keep the capillaries open after a 10% topload, a tetraPEGylated version of human Hb may also exhibit similar properties. Based on the results of the present study, Cys-13(β), or Cys-13(α), or Cys-111(α), or Cys-130(α), or a combination of these mutations could be engineered into human Hb to yield new homogeneous preparations of tetraPEGylated and hexaPEGylated Hb. Design of such Cys mutants of Hb as substrates for maleimide chemistry based PEGylation also provides an opportunity to engineer additional mutations to modulate other properties of Hb, i.e. reduced rate of auto-oxidation and increased stability.

### REFERENCES

Acharya A. S., Intaglietta M., Tsai A. G., Malavalli A, Vandegriff K., Winslow R. M., Smith P. K., Friedman J. M., and Manjula B. N. 2003. (PEG5K)₆-Hb: A non-hypertensive hemoglobin molecule generated by conservative PEGylation. Abstract, The 9^{th} International Symposium on Blood Substitutes, Tokyo, Japan, March 2003.
Acharya, A. S., Manjula, B N., and Smith, P. K. Hemoglobin crosslinkers. U.S. Pat. No. 5,585,484, issued Dec. 17, 1996.
Kerger, H., D. J. Saltzman, M. D. Menger, K. Messmer, and M. Intaglietta. 1996. Systemic and subcutaneous microvascular PO₂ dissociation during 4-h hemorrhagic shock in conscious hamsters. Am. J. Physiol. 270: H827-36.
Manjula B. N. and Acharya A. S. 2003. Purification and Molecular Analysis of Hemoglobin by High-Performance Liquid Chromatography. In Methods in Molecular Medicine: Hemoglobin Disorders: Molecular Methods and Protocols. Ed. Nagel R. L. Humana Press, Totowa, N.J. Vol. 82, p 31-47.
Manjula, B. N., Tsai, A., Upadhya, R., Perumalsamy, K., Smith, P. K., Malavalli, A, Vandegriff, K. D., Winslow, R. M., Intaglietta, M., Prabhakaran, M., Friedman, J. M., and Acharya, A. S. 2003. Site-specific PEGylation of hemoglobin at Cys-93(b): Correlation between the colligative properties of the PEGylated protein and the length of the conjugated PEG chain. Bioconjugate Chem. 14: 464-472.
Mirhashemi, S., G. A. Breit, R. H. Chavez, and M. Intaglietta. 1988. Effects of hemodilution on skin microcirculation. Am. J. Physiol. (Heart Circ. Physiol. 23) 254: H411-6.
Motterlini, R., K. D. Vandegriff, and R. M. Winslow. 1996. Hemoglobin-nitric oxide interactions and its implications. Transfusion Medicine Rev. 10:77-84.
Rohlfs, R. J., E. Bruner, A. Chiu, A. Gonzales, M. L. Gonzales, M. D. Magde, K. D. Vandegriff, and R. M. Winslow. 1998. Arterial blood pressure responses to cell-free hemoglobin solutions and the reaction with nitric oxide. J. Biol. Chem. 273:12128-34.
Tsai, A. G., B. Friesenecker, and M. Intaglietta. 1995. Capillary flow impairment and functional capillary density. Int. J. Microcirc. Clin. Exp. 15:238-43.
Tsai, A. G., B. Friesenecker, M. McCarthy, H. Sakai, and M. Intaglietta. 1998. Plasma viscosity regulates capillary perfusion during extreme hemodilution in hamster skinfold model. Am. J. Physiol. 275: H2170-80.
Tsai A G, Intaglietta M. 2001. High viscosity plasma expanders: Volume restitution fluids for lowering the transfusion trigger. Biorheology 38(2-3): 229-37.
Tsai, A., H. Kerger, and M. Intaglietta. 1996. Microvascular oxygen distribution: Effects due to free hemoglobin in plasma. In Blood Substitutes. New Challenges. R. M. Winslow, K. D. Vandegriff, and M. Intaglietta, editors. Birkhauser, Boston. 124-131.
Vandegriff K. D., Malavalli A, Wooldridge J., Lohman J., and Winslow R. M. 2003. MP4, a new nonvasoactive PEG-Hb conjugate. Transfusion 43:509-16.
Vandegriff, K. D., Rohlfs, R. J., and Winslow, R. M. 1997. Colloidal osmotic effects of hemoglobin based oxygen carriers. In Advances in Blood Substitutes: Industrial Opportunities and Medical Challenges. Eds. Winslow, R. M., Vandegriff, K. D., and Intaglietta, M., Birkhauser, Boton, pp 207-227.
Winslow, R. M. 1999. New transfusion strategies: red cell substitutes. Ann. Rev. Med. 50:337-53.

1. A PEGylated hemoglobin comprising a maleimide polyethylene glycol (PEG) conjugated to a thiol moiety of a cysteine residue of hemoglobins;
wherein the maleimide polyethylene glycol (PEG) is attached to a cysteine (Cys) residue at one or more of Cys-93(β), Cys-111(α), Cys-13(α), Cys-13(β), or Cys-130(α);
wherein a polyethylene glycol (PEG) chain is not conjugated to an ε-amino group of a lysine residue of hemoglobin;
wherein each globin chain of the hemoglobin is PEGylated; and
wherein the PEGylated hemoglobin does not cause constriction of arterioles.

2. (canceled)

3. The PEGylated hemoglobin of claim 1, wherein two, four, six or eight maleimide polyethylene glycol (PEG) groups are attached to the hemoglobin.

4. The PEGylated hemoglobin of claim 1, wherein four maleimide polyethylene glycol (PEG) groups are attached to the hemoglobin.

5. (canceled)

6. The PEGylated hemoglobin of claim 1, wherein the maleimide polyethylene glycol (PEG) is a maleimide phenyl polyethylene glycol (PEG).

7. The PEGylated hemoglobin of claim 1, comprising a PEG with a molecular weight of 200-40,000 daltons.

8. The PEGylated hemoglobin of claim 1, wherein the polyethylene glycol (PEG) has a molecular weight of 3,000-5,000 daltons.

9. The PEGylated hemoglobin of claim 1, wherein the polyethylene glycol (PEG) has a molecular weight of 5,000 daltons.

10. The PEGylated hemoglobin of claim 1, wherein PEGylation does not alter the charge at the site of attachment to hemoglobin of the maleimide polyethylene glycol (PEG).

11. (canceled)

12. The PEGylated hemoglobin of claim 1, wherein a δ-mercapto butyrimidyl chain or a γ-mercapto propylamide chain does not link the polyethylene glycol (PEG) to the hemoglobin.

13. The PEGylated hemoglobin of claim 1, which is homogeneous both in terms of molecular size and site of PEGylation.

14. The PEGylated hemoglobin of claim 1, having a radius of 5-6 nm.

15. (canceled)

16. The PEGylated hemoglobin of claim 1, wherein the hemoglobin is of human, canine, chicken, sheep, murine, or feline origin.

17. A method of preparing a PEGylated hemoglobin comprising conjugating a maleimide polyethylene glycol (PEG) to a thiol moiety of a cysteine residue of hemoglobin;
wherein the maleimide polyethylene glycol (PEG) is attached to a cysteine (Cys) residue at one or more of Cys-93(β), Cys-111(α), Cys-13(α), Cys-13(β), or Cys-130(α);
wherein each globin chain of the hemoglobin is PEGylated; and
wherein the method is carried out without the use of a thiolating compound for introduction of new thiol groups on an ε-amino group of a surface lysine residue of hemoglobin.

18. (canceled)

19. The method of claim 17, wherein two, four, six or eight maleimide polyethylene glycol (PEG) groups are attached to the hemoglobin.

20. The method of claim 17, wherein four maleimide polyethylene glycol (PEG) groups are attached to the hemoglobin.

21. (canceled)

22. The method of claim 17, wherein the maleimide polyethylene glycol (PEG) is a maleimide phenyl polyethylene glycol (PEG).

23. The method of claim 17, comprising a PEG with a molecular weight of 200-40,000 daltons.

24. The method of claim 17, wherein the polyethylene glycol (PEG) has a molecular weight of 3,000-5,000 daltons.

25. The method of claim 17, wherein the polyethylene glycol (PEG) has a molecular weight of 5,000 daltons.

26. The method of claim 17, wherein PEGylation does not alter the charge at the site of attachment to hemoglobin of the maleimide polyethylene glycol (PEG).

27. (canceled)

28. The method of claim 17, wherein an extension arm is not used to link the polyethylene glycol (PEG) to the hemoglobin.

29. The method of claim 17, wherein the hemoglobin is of human, canine, chicken, sheep, murine, or feline origin.

30. A PEGylated hemoglobin produced by the method of claim 17.

31. A composition comprising the PEGylated hemoglobin of claim 1 and a pharmaceutically acceptable carrier.

32. A blood substitute comprising the PEGylated hemoglobin of claim 1.

33. A method of treating a subject which comprises administering to the subject the blood substitute of claim 32.

34-36. (canceled)
